(19) United States
(12) Patent Application Publication
(10) Pub. No.: US 2023/0225762 A1
(43) Pub. Date: Jul. 20, 2023

(54) DEVICES, SYSTEMS, AND METHODS FOR FALLOPIAN TUBE ACCESS, EXPANSION, AND PROTECTION DURING MEDICAL EVALUATIONS AND TREATMENTS

(71) Applicant: Covidien LP, Mansfield, MA (US)

(72) Inventors: Lisa M. DeStefano, Eliot, ME (US); Alaena D. Maiorano, Huntington Beach, CA (US)

(21) Appl. No.: 17/577,613

(22) Filed: Jan. 18, 2022

(51) Int. Cl. A61B 17/42; A61B 17/34

(52) U.S. Cl. CPC A61B 17/42; A61B 17/3423; A61B 17/435; A61B 2017/4233

(57) ABSTRACT

A fallopian tube access system includes an outer tube assembly including an outer tube and a sleeve extending distally from the outer tube, and an inner tube assembly including an inner tube and an expander extending distally from the inner tube. The inner tube assembly is positioned within the outer tube assembly such that the expander is located within the sleeve in a compressed state. The outer tube assembly is movable relative to the inner tube assembly to expose the expander and transition the expander from the compressed state to an expanded state.

This disclosure generally relates to medical devices, systems, and methods of use. More particularly, this disclosure relates to devices, systems, and methods for accessing, expanding, and protecting fallopian tubes during medical evaluations and treatments.

### BACKGROUND

Fallopian tubes are accessed for examination or performance of medical procedures, such as evaluating infertility, cell sampling for tubular malignancy, or treating tubal pregnancy. To gain access to a fallopian tube, a catheter may be inserted through the cervix, into the uterus, and positioned adjacent the opening of the fallopian tube. A flexible medical instrument can then be passed through the catheter and into the fallopian tube. It can be difficult, however, to properly position the catheter relative to the fallopian tube and/or to pass the medical instrument through the fallopian tube. Accordingly, diagnosis and treatment of the fallopian tubes may be hampered by the difficulty in accessing the interior of the fallopian tubes in a minimally invasive manner.

### SUMMARY

This disclosure describes devices, systems, and methods for accessing, expanding, and protecting a fallopian tube. The devices, systems, and methods enable a fallopian tube to be expanded, straightened as necessary, and accessed during a medical evaluation or treatment (e.g., a surgical procedure) while minimizing or preventing injury to the fallopian tube, and may be utilized with a variety of medical or surgical instruments and/or fluids, such as those utilized for, for example, visualization, biopsy, and/or therapeutic or interventional treatments. The devices, systems, and methods provide suitable clearance in a fallopian tube for access and removal of diagnostic and/or treatment devices, and reduce the risk of tubal damage during medical procedures.

In aspects, this disclosure provides a fallopian tube access system including an outer tube assembly and, as needed for procedures, an inner tube assembly. The outer tube assembly includes an outer tube and a sleeve extending distally from the outer tube, and the inner tube assembly includes an inner tube and an expander extending distally from the inner tube. The inner tube assembly is positioned within the outer tube assembly such that the expander is located within the sleeve in a compressed state. The outer tube assembly is movable relative to the inner tube assembly to expose the expander and transition the expander from the compressed state to an expanded state.

In some aspects, the outer tube assembly further includes a luer connector coupled to a proximal end portion of the outer tube. In certain aspects, the outer tube includes a strain relief in the proximal end portion adjacent the luer connector.

The sleeve may have a fixed inner diameter. The sleeve may include an atraumatic distal tip including a curved leading edge and a chamfered surface interconnecting the curved leading edge and an inner wall of the sleeve.

In some aspects, the expander includes a transition region coupled to the inner tube and a body extending distally from the transition region. In certain aspects, the body of the expander has an atraumatic distal tip. In some aspects, when the expander is in the expanded state, the transition region has a variable inner diameter and the body has a uniform inner diameter. The expander may include a wound wire positioned between smooth, flexible inner and outer polymeric sheets. In some aspects, the wound wire is heat set to an expanded inner diameter corresponding to the expanded state. In some aspects, the expander includes a body having first and second longitudinal sides that overlap one another. In certain aspects, an amount of overlap between the first and second longitudinal sides is greater when the expander is in the compressed state than when the expander is in the expanded state.

At least one of the sleeve or the expander may include an imaging marker.

In aspects, this disclosure provides a method of accessing a fallopian tube that includes: inserting a sleeve of a fallopian tube access system into a fallopian tube, the sleeve extending distally from an outer tube of an outer tube assembly of the fallopian tube access system, the sleeve containing an expander loaded therein in a compressed state, the expander extending distally from an inner tube of an inner tube assembly of the fallopian tube access system; and moving the outer tube assembly of the fallopian tube access system proximally relative to the inner tube assembly to retract the sleeve relative to the expander so that the expander transitions from the compressed state to an expanded state within the fallopian tube.

The method may further include: passing a medical instrument through a lumen defined in the inner tube and into a lumen defined in the expander; and performing a medical procedure in the fallopian tube with the medical instrument.

The method may further include moving the inner tube assembly of the fallopian tube access system proximally relative to the outer tube assembly to re-insert the expander into the sleeve.

In some aspects, inserting the sleeve includes positioning the sleeve within an interstitium and an isthmus of the fallopian tube.

In some aspects, the medical instrument is an ectopic pregnancy treatment device including an end effector assembly having a transport capsule for a gestational sac, and performing the medical procedure includes securing a gestational sac, which is located within the fallopian tube, inside of the transport capsule of the ectopic pregnancy treatment device. In certain aspects, the method further includes moving the expander of the fallopian tube access system, with the transport capsule of the ectopic pregnancy treatment device contained completely within the expander, out of the fallopian tube and into a uterus. In certain aspects, the method further includes: moving the fallopian tube access system proximally relative to the transport capsule of the ectopic pregnancy treatment device; and releasing the gestational sac from the transport capsule for implantation in the uterus.

The details of one or more aspects of this disclosure are set forth in the accompanying drawings and the description below. Other aspects, as well as features, objects, and advantages of the aspects described in this disclosure will be apparent from the description and drawings, and from the claims.

### BRIEF DESCRIPTION OF DRAWINGS

Various aspects of this disclosure are described hereinbelow with reference to the drawings, which are incorporated in and constitute a part of this specification, wherein:

FIG. 1 is a perspective view of a fallopian tube access system including an outer tube assembly and an inner tube assembly in accordance with aspects of the disclosure;

FIG. 2 is a cross-sectional view of the fallopian tube access system of FIG. 1, taken along section line 2-2 of FIG. 1;

FIG. 3 is a close-up view of the area of detail 3 in FIG. 2;

FIG. 4 is close-up view of the area of detail 4 in FIG. 2;

FIG. 5A is a side, partial cut-away view of the inner tube assembly of FIG. 1, shown with an expander of the inner tube assembly in a compressed state;

FIG. 5B is a side, partial cut-away view of the inner tube assembly of FIG. 1, shown with an expander of the inner tube assembly in an expanded state;

FIG. 6 is a cross-sectional view of the fallopian tube access system, taken along section line 6-6 of FIG. 2;

FIG. 7A is a cross-sectional view of the outer tube assembly of the fallopian tube access system of FIG. 1 positioned within a fallopian tube independent of the inner tube assembly in accordance with aspects of the disclosure;

FIG. 7B is a cross-sectional view of the fallopian tube access system of FIG. 1 positioned within a fallopian tube, showing the retraction of a sleeve of the outer tube assembly relative to an expander of the inner tube assembly;

FIG. 8 is a cross-sectional view of the fallopian tube access system and the fallopian tube of FIG. 7B, showing the expander in an expanded state after full retraction of the sleeve relative to the expander;

FIG. 9 is a cross-sectional view of the fallopian tube access system and the fallopian tube of FIG. 8, taken along section line 9-9 of FIG. 8;

FIG. 10 is a perspective view of the inner tube assembly of FIG. 8, shown with the expander in the expanded state;

FIG. 11 is a cross-sectional view of the fallopian tube access system and the fallopian tube of FIG. 8, showing the expander being pulled proximally relative to the sleeve for re-insertion of the expander into the sleeve;

FIG. 12 is an illustration of reproductive anatomy of a pregnant human female, showing the fallopian tube access system of FIG. 1 inserted through a vagina, a cervix and a uterus, and into a fallopian tube adjacent a gestational sac;

FIG. 13 is a cross-sectional view of the fallopian tube access system of FIG. 12, showing the expander in the expanded state of FIG. 8 and an end effector assembly of an ectopic pregnancy treatment device located within the expander in a collapsed condition;

FIG. 14 is a cross-sectional view of the fallopian tube access system and the ectopic pregnancy treatment device of FIG. 13, showing the end effector assembly of the ectopic pregnancy treatment device in a deployed condition and capturing the gestational sac;

FIG. 15 is a cross-sectional view of the fallopian tube access system and the ectopic pregnancy treatment device of FIG. 14, showing the end effector assembly of the ectopic pregnancy treatment device and the gestational sac moved inside and held within the expander of the fallopian tube access system;

FIG. 16 is an illustration of the fallopian tube access system and the ectopic pregnancy treatment device of FIG. 15, showing the fallopian tube access system and the ectopic pregnancy treatment device removed from the fallopian tube and positioned within the uterus;

FIG. 17 is an illustration of the fallopian tube access system and the ectopic pregnancy treatment device of FIG. 16, showing the fallopian tube access system retracted proximally relative to the end effector assembly of the ectopic pregnancy treatment device; and

FIG. 18 is an illustration of the gestational sac transported from the fallopian tube to the uterus by the ectopic pregnancy treatment device of FIG. 17 and released for implantation in the uterus.

### DETAILED DESCRIPTION

Aspects of this disclosure will now be described in detail with reference to the drawing figures wherein like reference numerals identify similar or identical elements. Throughout this description, the term “proximal” refers to a portion of a structure, or component thereof, that is closer to an operator (whether a surgeon, other clinician, or a surgical robot), and the term “distal” refers to a portion of the structure, or component thereof, that is farther from the operator. It should be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

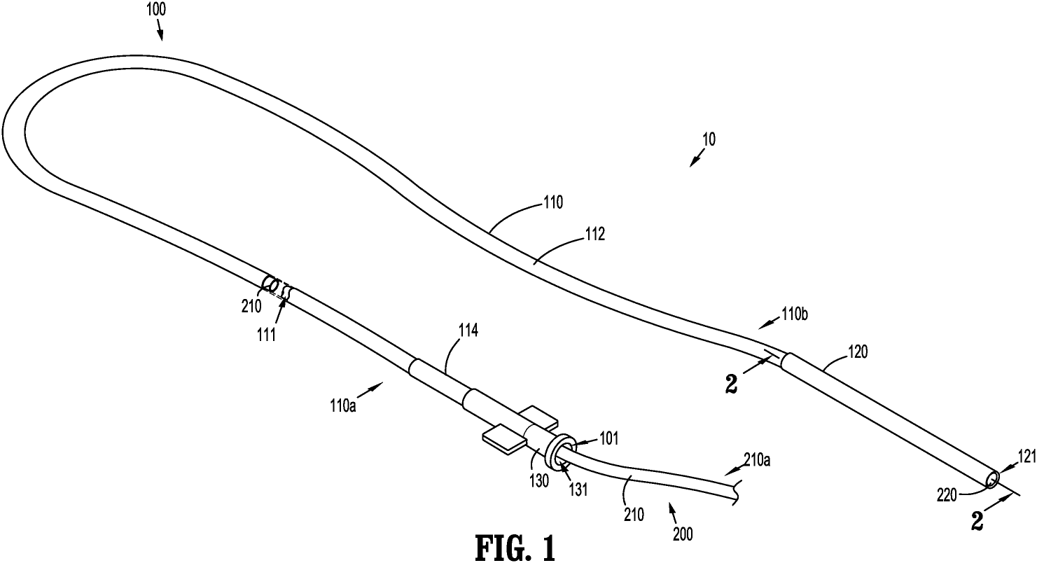

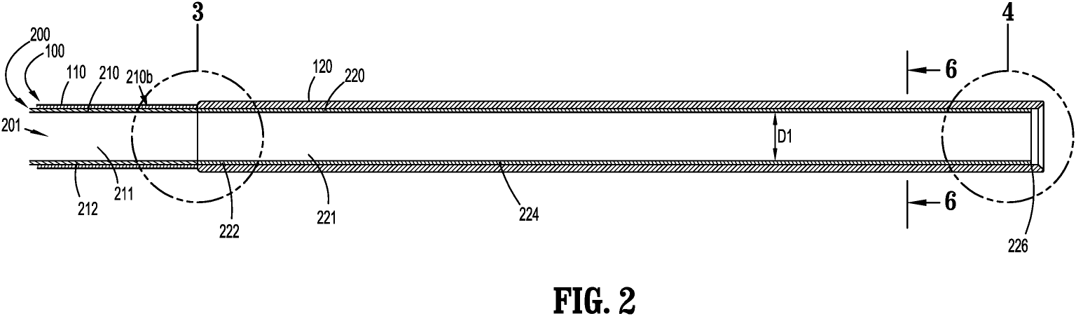

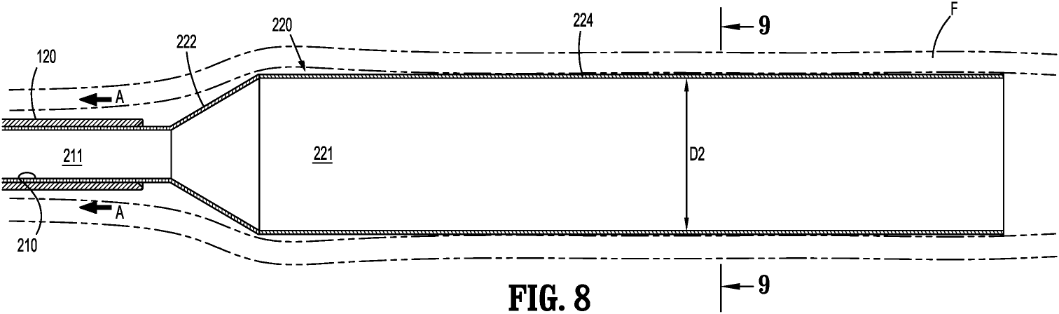

FIGS. 1 and 2 illustrate a fallopian tube access system 10 including an outer tube assembly 100 and, as procedurally necessary, an inner tube assembly 200 slidably disposed within the outer tube assembly 100. In an initial or undeployed configuration, as seen in FIGS. 1 and 2, an expander 220 (FIG. 2) of the inner tube assembly 200 is housed within a sleeve 120 of the outer tube assembly 100 in a compressed state or condition. The sleeve 120 and the expander 220 are axially movable relative to each other to deploy the expander 220 distally beyond the sleeve 120 so that the expander 220 is free to move to an expanded state or condition (see e.g., FIG. 8) and to retract the expander 220 back into the sleeve 120.

The outer tube assembly 100 includes an outer tube 110 and the sleeve 120 extending distally from the outer tube 110. The outer tube 110 includes an elongate body 112 defining a lumen 111 therethrough that is in fluid or open communication with a lumen 121 defined in the sleeve 120 thereby providing a continuous channel or conduit 101 extending through the outer tube assembly 100. The conduit 101 is configured to receive the inner tube assembly 200 therethrough. In aspects, the outer tube assembly 100 further includes a hub 130 extending proximally from the outer tube 110, and the hub 130 defines a lumen 131 therethrough that is constructed in open communication with the lumen 111 of the outer tube 110.

The hub 130 may be a connector (e.g., a luer connector) configured to engage a medical instrument (e.g., a syringe for flushing, a vacuum line for suctioning, etc.), and may act as a handle for gripping and manipulation by an operator. Additionally or alternatively, the hub 130 may include or be connectable to proximal end actuators (not shown) that can, for example, independently or simultaneously, advance, retract, hold, and/or release the outer and/or inner tube assemblies 100, 200.

The outer tube 110 is of sufficient length to enable the outer tube 110 to extend into a patient (e.g., through a body cavity) so that the hub 130 is located outside of the patient’s body and the sleeve 120 reaches a treatment site within the patient’s body. The sleeve 120 is of sufficient length to retain the entirety of the expander 220 of the inner tube assembly 200 therein.

The outer tube 110 and the sleeve 120 are each formed from any of a variety of biologically compatible polymeric materials. The outer tube 110 and the sleeve 120 have sufficient structural integrity (e.g., column strength or “pushability”) to permit the outer tube 110 and the sleeve 120 to be advanced to distal locations within a patient’s body without buckling or undesirable bending of the outer tube 110 and the sleeve 120. In aspects, the outer tube 110 is formed from a polymeric material having a braided construction. In some aspects, the outer tube 110 may have variable flexibility characteristics along its length (e.g., the outer tube 110 may be more flexible about a proximal end portion 110a of the outer tube 110 and less flexible towards a distal end portion 110b of the outer tube 110). Areas of higher and lower stiffness can be achieved by, for example, changing the polymer composition, changing the pitch and/or thickness of the braid, or thickness of wound wire, among other techniques within the purview of those skilled in the art. In aspects in which the hub 130 is secured to the outer tube 110, the outer tube 110 may further include a strain relief 114 in the proximal end portion 110a of the outer tube adjacent the hub 130. The strain relief 114 may be an area of lower stiffness and/or may be formed from an elastomeric material that reduces stresses on the outer tube 110 where it exits the hub 130.

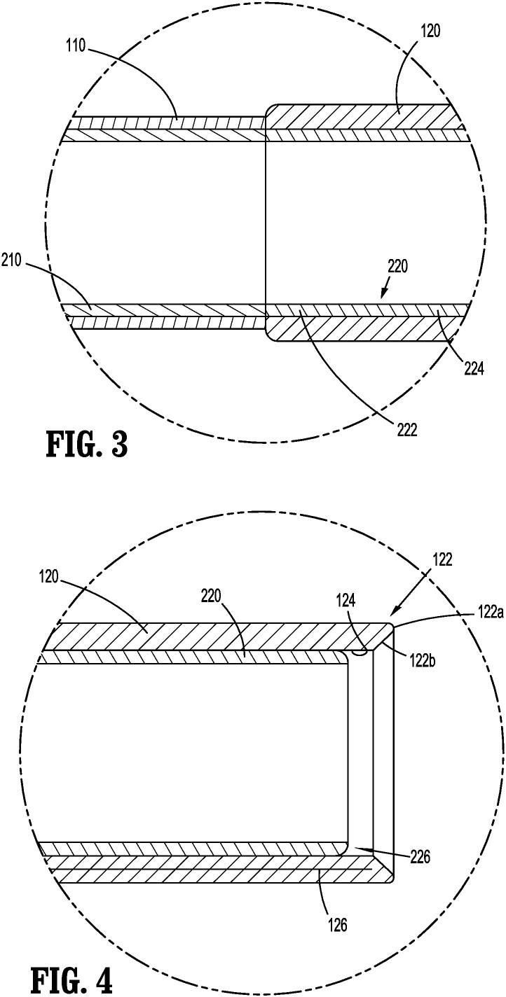

In aspects, the sleeve 120 is formed from a polymeric material that includes a torque and/or column strength enhancer, such as axially extending stiffening wires, spiral wrapped support layers, braided or woven reinforcement filaments, etc. The sleeve 120 is of sufficient strength to maintain its shape and integrity while housing the expander 220 in the sleeve 120, as well as during loading and deployment of the expander 220 into and out of the sleeve 120. Further, the ability to transmit torque is desirable, for example, to avoid kinking upon rotation and/or to assist in steering. In some aspects, as shown in FIG. 3, the outer tube 110 is bonded to the sleeve 120. In other aspects, the outer tube 110 and the sleeve 120 are integrally formed, with the sleeve further including the strength enhancer.

As shown in FIG. 4, the sleeve 120 includes an atraumatic distal tip 122 having a curved leading edge 122a and a chamfered or sloped surface 122b interconnecting the curved leading edge 122a and an inner wall 124 of the sleeve 120 to facilitate smooth deployment and retraction of the expander 220 relative to the sleeve 120. The sleeve 120 may include one or more markers 126 (e.g., radiopaque bands) for visibility under imaging (e.g., fluoroscopic monitoring) during advancement and placement of the outer tube assembly 100 within a patient.

With continued reference to FIGS. 1 and 2, the inner tube assembly 200 includes an inner tube 210 and an expander 220 extending distally from the inner tube 210. The inner tube 210 includes an elongate body 212 defining a lumen 211 therethrough that is in fluid or open communication with a lumen 221 defined in the expander 220 thereby providing a continuous channel or conduit 201 extending through the inner tube assembly 200. The conduit 201 is configured for delivery of medical devices and/or fluid (e.g., contrast media, dyes, medicaments, saline, etc.) therethrough.

The inner tube 210 is of sufficient length so that the expander 220 is positionable at a treatment site within a patient’s body and the inner tube 210 extends from the expander 220, through the outer tube 110, and proximally beyond the hub 130 of the outer tube assembly 100. A proximal end portion 210a of the inner tube 210 may be configured to advance, retract, hold, and/or release the inner tube assembly 200 and/or medical instruments coupled to or extending through the inner tube 210. The inner tube 210 is sized and shaped to freely slide inside of the conduit 101 of the outer tube assembly 100.

The inner tube 210 is formed from any of a variety of biologically compatible polymeric materials as described above with respect to the outer tube 110 and the sleeve 120 so that the inner tube 210 has sufficient structural integrity (e.g., column strength or “pushability”) to advance and retract the expander 220. In aspects, the inner tube 210 is formed from a polymeric material having a braided construction. In some aspects, the inner tube 210 may have variable flexibility characteristics along its length (e.g., the inner tube 210 may be more flexible about the proximal end portion 210a and less flexible towards a distal end portion 210b of the inner tube 210).

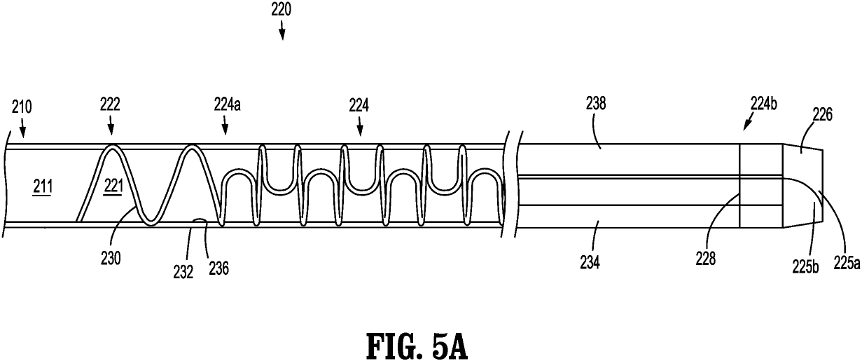

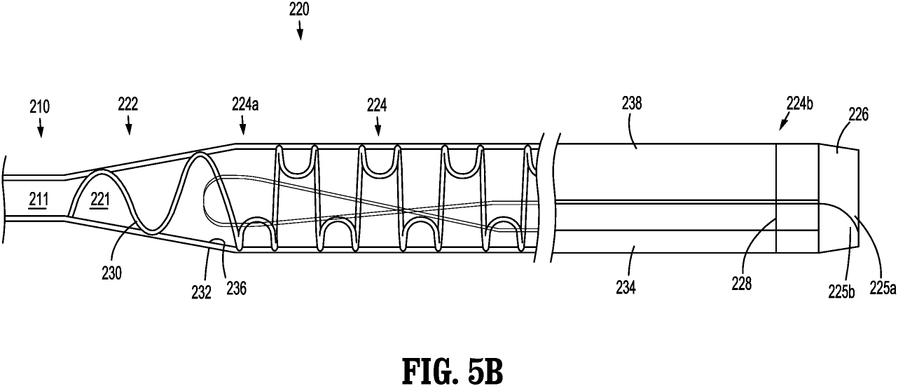

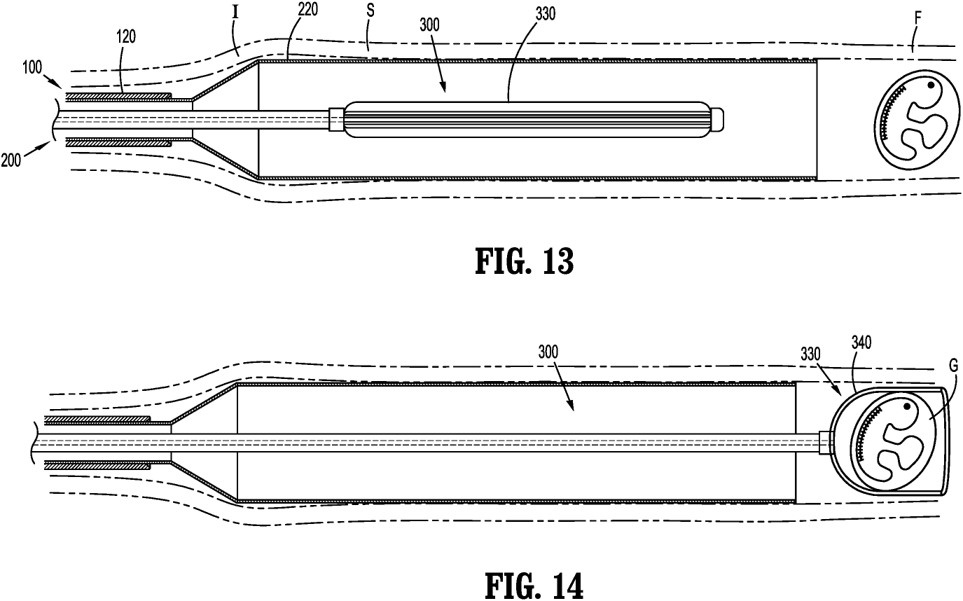

As shown in FIG. 5A and FIG. 5B, in conjunction with FIGS. 2-4, the expander 220 includes a transition region 222 coupled to and extending distally from the inner tube 210, and a body 224 extending distally from the transition region 222 to an atraumatic distal tip 226. The bond between the transition region 222 of the expander 220 and the inner tube 210 ensures secure attachment of the expander 220 to the inner tube 210, while maintaining patency of the inner tube 210, when pushing and pulling the expander 220 in and out of the sleeve 120 of the outer tube assembly 100 (e.g., the bond’s strength exceeds the forces to deploy or capture the expander 220 inside of the sleeve 120). The body 224 of the expander 220 may include one or more markers 228 (e.g., radiopaque bands) for visibility under imaging (e.g., fluoroscopic monitoring). In aspects, the markers 228 are placed at proximal and/or distal end portions 224a, 224b of the body 224. The atraumatic distal tip 226 of the expander 220 can have a variety of configurations such as, for example, tapered (as seen in FIGS. 5A and 5B), non-tapered (FIG. 13), slightly flared, etc., and may be formed from a low-durometer polymeric material.

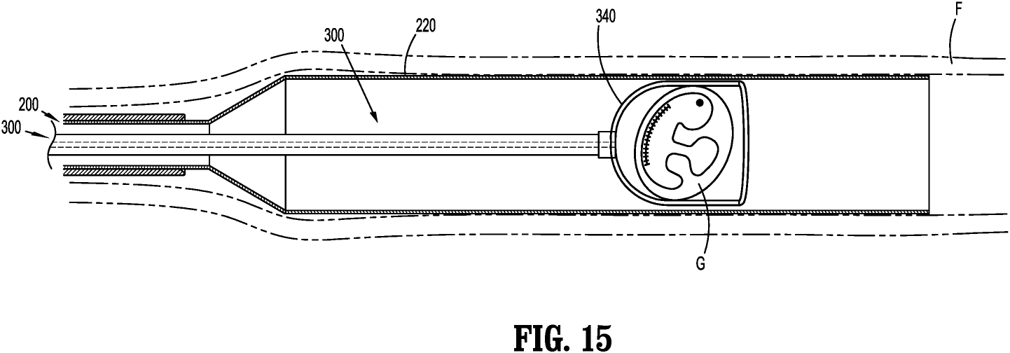

The expander 220 has a compressed state, as seen in FIG. 5A, (e.g., when the expander 220 is loaded within the sleeve 120 of the outer tube assembly 100) in which the body 224 has a first or compressed inner diameter “D1” (FIG. 2) and an expanded state, as seen in FIG. 5B, (e.g., when the expander 220 extends distally beyond the sleeve 120 of the outer tube assembly 100) in which the body 224 has a second or expanded inner diameter “D2” (FIG. 8) that is greater than the first inner diameter “D1.” In some aspects, the second inner diameter “D2” of the body 224 generally approximates an outer diameter of a medical instrument (e.g., an ectopic pregnancy treatment device 300 shown in FIG. 15).

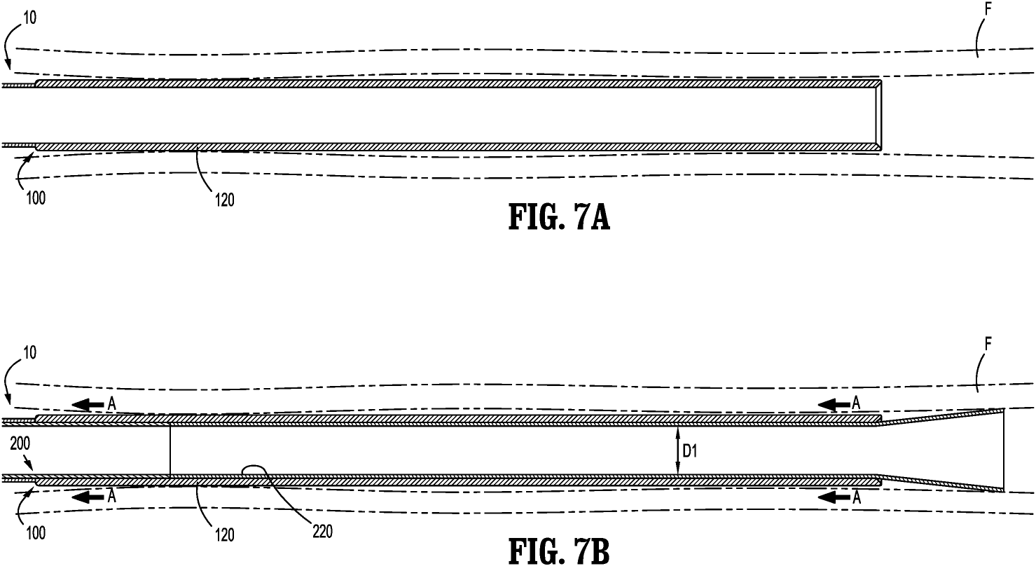

With continued reference to FIGS. 5A and 5B, the expander 220 is formed from a composite of biologically compatible materials. The expander 220 includes a wound wire 230 fused between smooth, flexible inner and outer polymeric sheets 232, 234. The wound wire 230 may be formed from a metal, such as nitinol or stainless steel, and in some aspects, the wound wire 230 is formed from a shape memory material. The inner polymeric sheet 232 may be formed from a low-friction material, such as a fluoropolymer (e.g., PTFE, FEP, PFA, and the like) and the outer polymeric sheet 234 may be formed from a variety of polymeric materials such as, for example, polyethylene, polypropylene, polyimide, polyamide, or the like, which may be compounded with a low-friction additive, such as siloxane. The inner polymeric sheet 232 forms an inner wall 236 of the expander 220 which defines the lumen 221 for receiving medical instruments and fluid therethrough, and the outer polymeric sheet 234 forms an outer wall 238 of the expander 220 that is configured for positioning adjacent an inner wall of a fallopian tube “F” (FIG. 7B). In aspects, the wound wire 230 is heat-set at the second inner diameter “D2” (FIG. 8) and compressed within the sleeve 120 of the outer tube assembly 100 to the first inner diameter “D1” (FIG. 7B) such that the expander 220 self-expands to the second inner diameter “D2” when removed from the sleeve 120.

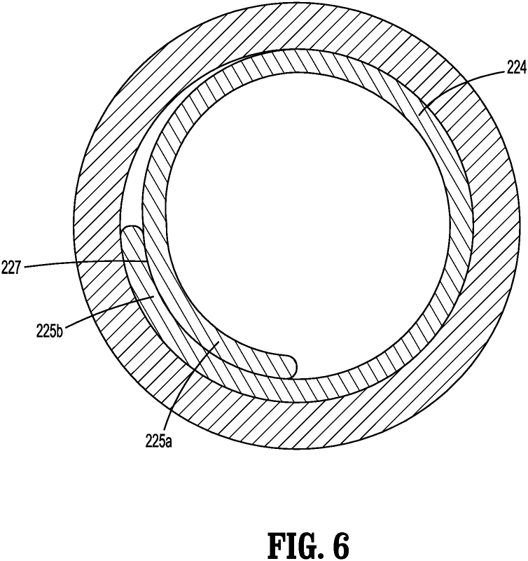

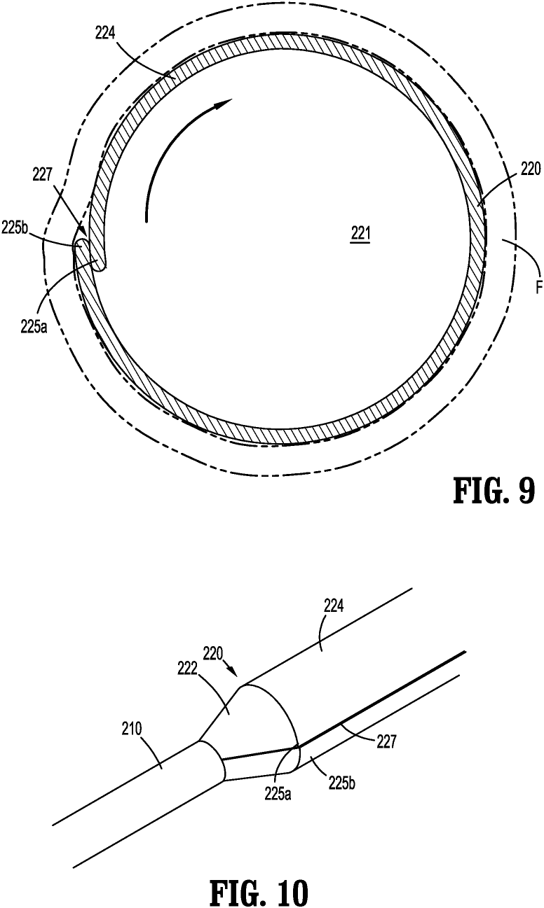

As shown in FIGS. 5A, 5B, and 6, the body 224 of the expander 220 includes first and second longitudinal sides 225a and 225b that overlap one another and define a longitudinal slit 227 therebetween. The amount of overlap between the longitudinal sides 225a, 225b is greater when the expander 220 is in the compressed state (FIG. 6) than in the expanded state (FIG. 9). In aspects, the transition region 222 of the expander 220 folds upon itself and the body 224 is rolled upon itself during insertion into the sleeve 120 of the outer tube assembly 100. The compressed state within the sleeve 120 provides the radial strength necessary to open a fallopian tube and maintain placement of the expander 220 in the expanded state within the fallopian tube under compressive stresses (e.g., during a muscle spasm or to straighten a tortuous section of the fallopian tube). It should be understood that the radial force and configuration of the expander 220 may vary depending upon, for example, the wire size, braided wire density, braid pattern, etc.

In methods of preparing the fallopian tube access system 10 for use, the outer tube assembly 100 can be used as a primary access lumen, without the inner tube assembly 200, or used in conjunction with the inner tube assembly 100. In a method of using the outer tube assembly 100 independently of the inner tube assembly 200, the outer tube assembly 100, in the configuration seen in FIG. 1, is inserted into a fallopian tube “F”, as shown in FIG. 7A. Imaging may be utilized during insertion of the outer tube assembly 100 into the fallopian tube “F” for guidance and placement. As seen in FIG. 7A, once in the desired position within the fallopian tube “F,” medical instruments and/or fluids may be delivered into and out of the lumen 111 of the outer tube 110. The sleeve 120 thus protects the inner wall of the fallopian tube “F” from damage, such as potential scarring from medical instruments that would otherwise directly transverse the fallopian tube “F.”

In a method preparing the fallopian tube access system 10 for use, namely the mated outer tube and inner tube assemblies 100, 200, the proximal end portion 210a of the inner tube 210 of the inner tube assembly 200 is inserted proximally into the lumen 121 of the sleeve 120 of the outer tube assembly 100, and pushed through the lumen 111 of the outer tube 110 and the lumen 131 of the hub 130 until the proximal end portion 210a exits the hub 130. Once the proximal end portion 210a of the inner tube 210 extends proximally beyond the hub 130, the proximal end portion 210a of the inner tube 210 may be pulled proximally to load the expander 220 into the sleeve 120 of the outer tube assembly 100 in the compressed state, as seen in FIGS. 1 and 2, so that the fallopian tube access system 10 is in the initial configuration and ready for use.

In a method of use, the fallopian tube access system 10, in the initial configuration seen in FIGS. 1 and 2, is inserted into a fallopian tube “F”, as shown in FIG. 7B. Imaging may be utilized during insertion of the fallopian tube access system 10 into the fallopian tube “F” for guidance and placement. As seen in FIG. 7B, once in the desired position within the fallopian tube “F,” the outer tube assembly 100 is moved proximally, in the direction of arrow “A,” relative to the inner tube assembly 200 to retract the sleeve 120 from over the expander 220 and expose the expander 220.

As shown in FIGS. 8-10, once the sleeve 120 is fully retracted proximally beyond the expander 220, the expander 220 expands to its expanded state within the fallopian tube “F.” The radial force of the expander 220 as it moves from the compressed state to the expanded state secures the expander 220 within the fallopian tube “F” and increases the inside diameter of the fallopian tube “F.” In this expanded state, medical instruments and/or fluids may be delivered through the lumen 211 of the inner tube 210, and into and out of the lumen 221 of the expander 220. The expander 220 thus protects the inner wall of the fallopian tube “F” from damage, such as potential scarring from medical instruments that would otherwise directly transverse the fallopian tube “F.”

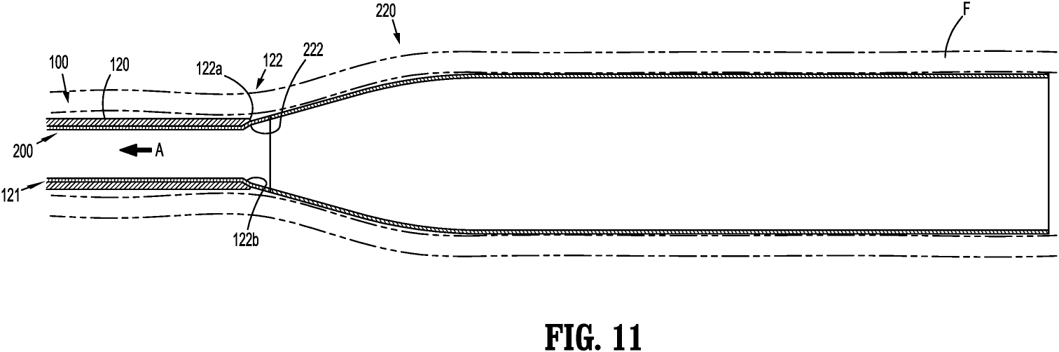

Once the medical procedure is complete, the inner tube assembly 200 is slid proximally relative to the outer tube assembly 100, in the direction of arrow “A,” as shown in FIG. 11. Additionally or alternative, the sleeve 120 may be slid distally over the expander 220. As the expander 220 is withdrawn proximally into the sleeve 120, the transition region 222 first contacts the curved leading edge 122a of the distal tip 222 of the sleeve 120 and follows the chamfered surface 122b into the lumen 121 of the sleeve 120 which helps facilitate retraction of the expander 220 and aids in compressing the expander 220 into the sleeve 120. Further movement of the expander 220 into the sleeve 120 collapses the expander 220 back to its compressed state within the sleeve 120 and allows the fallopian tube “F” to return to its biased inside diameter. The fallopian tube access system 10 may then be removed from the fallopian tube “F.”

As discussed above, the fallopian tube access system 10 may be utilized with a variety of medical instruments. In aspects, the fallopian tube access system 10 is utilized with an ectopic pregnancy treatment device 300 (FIG. 13) to provide crush protection for an embryo during transport from the fallopian tube into the uterus for implantation. For a detailed description of the structure and function of the ectopic pregnancy treatment device, reference may be made to U.S. Pat. Appl. Pub. No. 2021/0236169, the entire contents of which is hereby incorporated by reference herein.

The devices, systems, and methods utilized for treating an ectopic pregnancy includes the atraumatic engagement of the devices and systems with a fallopian tube, as well as the atraumatic capture, transport, and release of a gestation sac from a location within the fallopian tube to a location within a uterus to enable implantation and continued growth of the embryo as the pregnancy processes. “Atraumatic” as utilized herein refers to an action or actions that avoid tubal damage and maintain viability of the gestational sac (including the embryo and other matter therein) without inflicting irreversible damage thereupon.

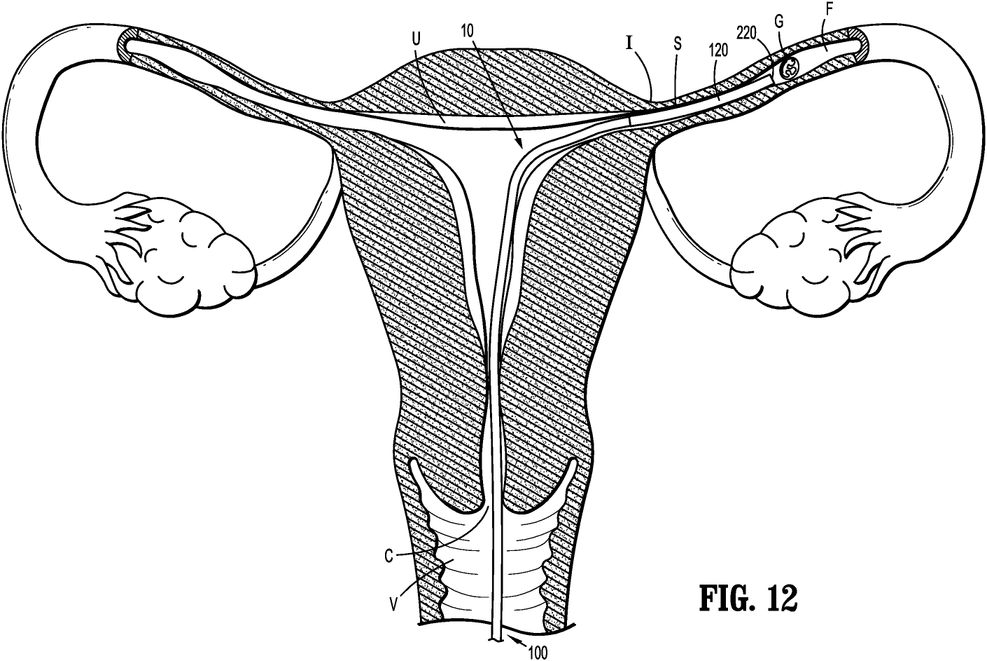

In a method of use for treating an ectopic pregnancy without compromising the pregnancy, shown in FIG. 12, the fallopian tube access system 10, in the initial configuration seen in FIG. 1, is inserted, with the sleeve 120 leading, trans-vaginally and tracked under imaging guidance through the vagina “V”, the cervix “C”, and the uterus “U”, and into the fallopian tube “F.” Given its column strength, the sleeve 120 can be pushed through smaller diameter (e.g., the interstitium “I” and the isthmus “S”) and/or obstructed areas (as encountered) of the fallopian tube “F” to a position adjacent a gestational sac “G.” In some aspects, the atraumatic distal tip 226 (FIG. 5A) of the expander 220 may be slightly exposed by moving the sleeve 120 proximally relative to the expander 220 and locking this position in place before pushing the fallopian tube access system 10 into the fallopian tube “F.”

Once properly positioned at its intended location, the sleeve 120 is removed completely from the expander 220 by pulling the outer tube assembly 100 proximally and allowing the expander 220 to self-expand radially at least within the interstitium “I” and isthmus “S” of the fallopian tube “F,” as shown in FIG. 13. The sleeve 120 may be moved to the uterus “U” (FIG. 12) or remain adjacent the expander 220 to help support the inner tube assembly 200. The expander 220 provides an adequate radial force to enlarge the interstitium “I” and the isthmus “S,” and ensures patency through the fallopian tube’s tortuous, bent, or twisted sections and adequate clearance if muscular spasms are experienced during the medical procedure.

The ectopic pregnancy treatment device 300 is then inserted into the proximal end portion 210a (FIG. 1) of the inner tube 210 and fed through the inner tube assembly 200 so that the ectopic pregnancy treatment device 300 can be deployed into the fallopian tube “F” through the expander 220. In some aspects, a funnel-type attachment (not shown) may be provided at the proximal end portion 210a of the inner tube 210 to help align and load the ectopic pregnancy treatment device 300 into the lumen 211 of the inner tube 210. Alternatively, the ectopic pregnancy treatment device 300 may be pre-loaded inside of the inner tube assembly 200 of the fallopian tube access system 10 prior to insertion of the fallopian tube access system 10 into the patient such that, once the expander 220 is expanded, the ectopic pregnancy treatment device 300 can be deployed.

An end effector assembly 330 of the ectopic pregnancy treatment device 300 is passed into the expander 220, as seen in FIG. 13. The end effector assembly 330 is then transitioned from a collapsed condition seen in FIG. 13 to an expanded, inverted condition shown in FIG. 14. Prior, after, overlapping, or simultaneously with the application of vacuum through the ectopic pregnancy treatment device 300, the end effector assembly 330 is advanced distally towards the gestational sac “G” such that, as a result of this distal movement and the applied vacuum establishing a transport cavity 340 within the end effector assembly 330, the gestational sac “G” is atraumatically drawn into and captured within the transport cavity 340. Alternatively, the end effector assembly 330 may not be moved distally but held substantially stationary during the application of vacuum. The gestational sac “G” may be fully received within the internal volume of the transport cavity 340 or may be partially received therein. The volume defined by the transport cavity 340 may be set based on the size of the gestational sac “G” and/or the amount of encapsulation desired within the transport cavity 340.

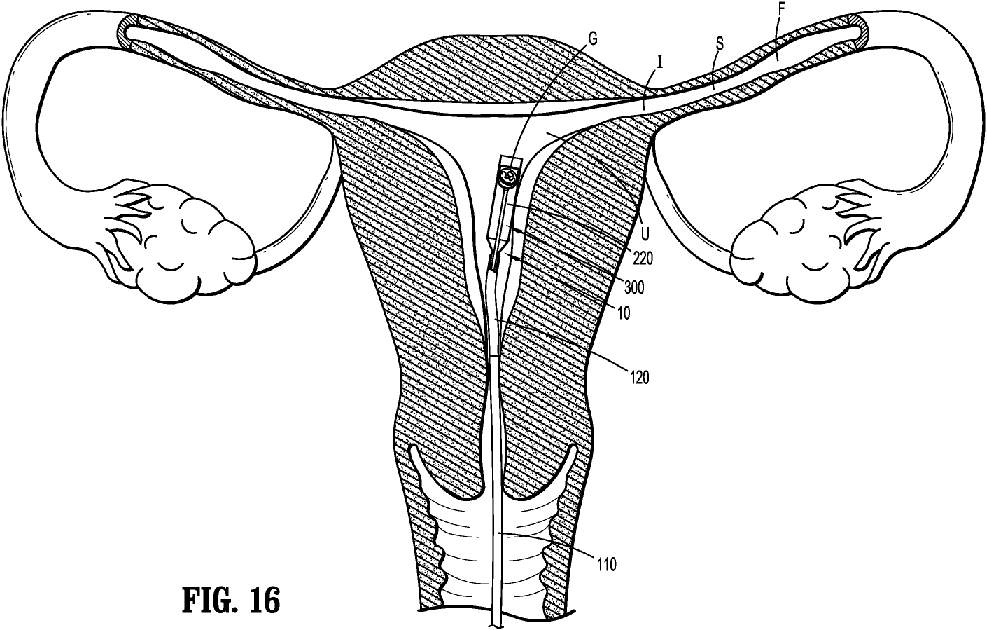

Once the gestational sac “G” is contained within the transport cavity 340, the transport cavity 340 is moved completely inside of the expander 220, as shown in FIG. 15. The inner tube assembly 200 and the ectopic pregnancy treatment device 300 may be locked in place (e.g., by proximal actuators on the inner tube assembly 200) so that the inner tube assembly 200 and the ectopic pregnancy treatment device 300 may be moved simultaneously and without impediment, out of the fallopian tube “F” and into the uterus “U,” as shown in FIG. 16. The transport cavity 340 and the expander 220 protect the gestational sac “G″ during transport to the uterus U,” including urging through the smaller-diameter openings of the isthmus “S” and the interstitium “I,” navigating along tortuous paths, etc.

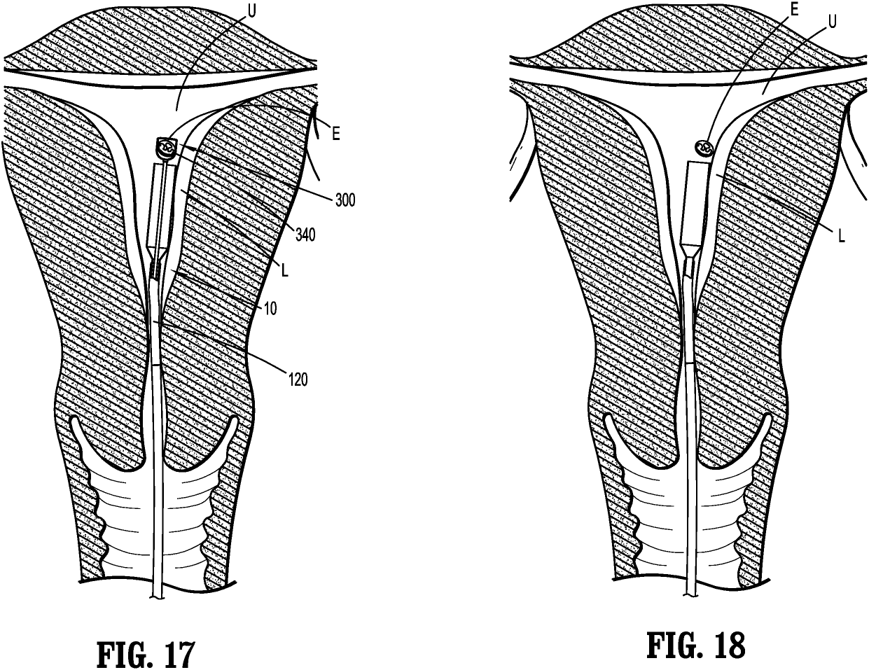

Once in the uterus “U,” the fallopian tube access system 10 is moved proximally relative to the transport capsule 340 (e.g., by releasing the proximal actuators and pulling the fallopian tube access system 10 proximally relative to the ectopic pregnancy treatment device 300) so that the transport capsule 340 containing the embryo “E” is exposed within the uterus “U,” as shown in FIG. 17. Via imaging, the transport capsule 340 is placed adjacent the endometrial lining “L” of the uterus “U” and the embryo “E” is safely released (e.g., by release of the vacuum), for intended implantation, as shown in FIG. 18. The end effector assembly 330 of the ectopic pregnancy treatment device 300 may be returned to its collapsed condition and the expander 220 of the fallopian tube access system 10 may be returned to its compressed state. Both the ectopic pregnancy treatment device 300 and the fallopian tube access system 10 may be removed from the uterus “U” and trans-vaginally withdrawn from the patient.

While illustrated and described as being handheld devices and systems, it is contemplated and within the scope of this disclosure for the devices and systems to be configured to be detachably coupleable to and controllable by a robotic surgical system, such as the robotic surgical system shown and described in U.S. Pat. Appl. Pub. No. 2012/0116416, the entire contents of which are incorporated herein by reference.

It should be understood that to treat diverse patient anatomies and conditions, the expander may come in a variety of configurations (e.g., lengths, expanded inner diameters, etc.) desired for the treatment location and use (e.g., having a length sufficient to span a tortuous area of a fallopian tube, and compressed and expanded inner diameters to ensure patency and clearance of particular medical device(s) therethrough), and the sleeve is sized to accommodate the variety of expander lengths and compressed diameters.

While aspects of this disclosure are discussed in terms of devices, systems, and methods for accessing a fallopian tube, it is envisioned that the principles of this disclosure are equally applicable to devices, systems, and methods for accessing a range of hollow anatomical structures including, for example, veins, arteries, gastric structures, coronary structures, pulmonary structures, tubular structures such as those associated with reproductive organs, and the like.

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Additionally, the elements and features shown and described in connection with certain aspects of the disclosure may be combined with the elements and features of certain other aspects without departing from the scope of the disclosure, and that such modifications and variation are also included within the scope of the disclosure. Therefore, the above description should not be construed as limiting, but merely as exemplifications of aspects of the disclosure. Thus, the scope of the disclosure should be determined by the appended claims and their legal equivalents, rather than by the examples given.

What is claimed is:

1. A fallopian tube access system comprising:
an outer tube assembly including an outer tube and a sleeve extending distally from the outer tube; and
an inner tube assembly including an inner tube and an expander extending distally from the inner tube, the inner tube assembly positioned within the outer tube assembly such that the expander is located within the sleeve in a compressed state, the outer tube assembly being movable relative to the inner tube assembly to expose the expander and transition the expander from the compressed state to an expanded state.

2. The fallopian tube access system of claim 1, wherein the outer tube assembly further includes a luer connector coupled to a proximal end portion of the outer tube.

3. The fallopian tube access system of claim 2, wherein the outer tube includes a strain relief in the proximal end portion adjacent the luer connector.

4. The fallopian tube access system of claim 1, wherein the sleeve has a fixed inner diameter.

5. The fallopian tube access system of claim 1, wherein the sleeve includes an atraumatic distal tip including a curved leading edge and a chamfered surface interconnecting the curved leading edge and an inner wall of the sleeve.

6. The fallopian tube access system of claim 1, wherein the expander includes a transition region coupled to the inner tube and a body extending distally from the transition region.

7. The fallopian tube access system of claim 6, wherein the body of the expander has an atraumatic distal tip.

8. The fallopian tube access system of claim 6, wherein, when the expander is in the expanded state, the transition region has a variable inner diameter and the body has a uniform inner diameter.

9. The fallopian tube access system of claim 1, wherein the expander includes a wound wire positioned between smooth, flexible inner and outer polymeric sheets.

10. The fallopian tube access system of claim 9, wherein the wound wire is heat set to an expanded inner diameter corresponding to the expanded state.

11. The fallopian tube access system of claim 1, wherein the expander includes a body having first and second longitudinal sides that overlap one another.

12. The fallopian tube access system of claim 11, wherein an amount of overlap between the first and second longitudinal sides is greater when the expander is in the compressed state than when the expander is in the expanded state.

13. The fallopian tube access system of claim 1, wherein at least one of the sleeve or the expander includes an imaging marker.

14. A method of accessing a fallopian tube, the method comprising:
inserting a sleeve of a fallopian tube access system into a fallopian tube, the sleeve extending distally from an outer tube of an outer tube assembly of the fallopian tube access system, the sleeve containing an expander loaded therein in a compressed state, the expander extending distally from an inner tube of an inner tube assembly of the fallopian tube access system; and
moving the outer tube assembly of the fallopian tube access system proximally relative to the inner tube assembly to retract the sleeve relative to the expander so that the expander transitions from the compressed state to an expanded state within the fallopian tube.

15. The method of claim 14, further including:
passing a medical instrument through a lumen defined in the inner tube and into a lumen defined in the expander; and
performing a medical procedure in the fallopian tube with the medical instrument.

16. The method of claim 14, further including:
moving the inner tube assembly of the fallopian tube access system proximally relative to the outer tube assembly to re-insert the expander into the sleeve.

17. The method of claim 14, wherein inserting the sleeve includes positioning the sleeve within an interstitium and an isthmus of the fallopian tube.

18. The method of claim 15, wherein the medical instrument is an ectopic pregnancy treatment device including an end effector assembly having a transport capsule for a gestational sac, and performing the medical procedure includes securing a gestational sac located in the fallopian tube inside of the transport capsule of the ectopic pregnancy treatment device.

19. The method of claim 18, further including:
moving the expander of the fallopian tube access system, with the transport capsule of the ectopic pregnancy treatment device contained completely within the expander, out of the fallopian tube and into a uterus.

20. The method of claim 19, further including:
moving the fallopian tube access system proximally relative to the transport capsule of the ectopic pregnancy treatment device; and
releasing the gestational sac from the transport capsule for implantation in a uterus lining of the uterus.
